(19) United States
(12) Patent Application Publication
(10) Pub. No.: US 2016/0030558 A1
(43) Pub. Date: Feb. 4, 2016

(54) ENHANCEMENT OF VACCINES

(71) Applicant: HEALTH RESEARCH, INC., Buffalo, NY (US)

(72) Inventors: Brahm H. SEGAL, Williamsville, NY (US); Scott ABRAMS, Amherst, NY (US); Kunle ODUNSI, Williamsville, NY (US); Melissa GRIMM, Clarence Center, NY (US); Nazmul H. KHAN, East Amherst, NY (US); Nonna KOLOMEYEVSKAYA, Brighton, MA (US); Kelly SINGEL, Buffalo, NY (US)

(21) Appl. No.: 14/774,763

(22) PCT Filed: Mar. 13, 2014
(86) PCT No.: PCT/US2014/025456

§ 371 Date: Sep. 11, 2015

Related U.S. Application Data
(60) Provisional application No. 61/778,762, filed on Mar. 13, 2013.

(51) Int. Cl. A61K 39/395; A61K 39/39; G01N 33/50; A61K 39/00

(52) U.S. Cl. CPC A61K 39/3955; A61K 39/0011; A61K 39/39; G01N 33/5011; A61K 2039/505; A61K 2039/55555; A61K 2039/5555; A61K 2039/55561; A61K 2039/57; A61K 2039/545; G01N 2500/10

(57) ABSTRACT

Provided is a method for enhancing the efficacy of cancer vaccines, such as tumor vaccines. The method involves administering to an individual who is in need of therapy for a tumor an anti-cancer agent and an agent that causes depletion of myeloid cells and/or inhibits recruitment of myeloid cells to the tumor. The effect of the anti-cancer agent on the tumor is greater relative to the effect of the anti-cancer agent in the absence of the anti-myeloid cell agent. Also provided is a method for identifying candidates for the therapy. This approach involves determining if an individual has a tumor characterized by undesirable myeloid cell proliferation and/or tumor infiltration and/or myeloid cell recruitment to the tumor, and if such determination is made, designating the individual as a candidate for the therapy. In one embodiment, the identification of the individual as such a candidate is followed by the therapeutic approach.

### CROSS REFERENCE TO RELATED APPLICATIONS

This application claims priority to U.S. application No. 61/778,762, filed Mar. 13, 2013, the disclosure of which is incorporated herein by reference.

### FIELD The present invention relates generally to therapeutic approaches to cancer and more specifically to enhancing the activity of cancer vaccines.

### BACKGROUND OF THE INVENTION

Epithelial ovarian cancer (EOC) is a significant medical problem in the U.S., and in many countries throughout the world. It is typically diagnosed at an advanced stage, and relapse of the disease occurs in the vast majority of patients, with a mean time of about 18 months after primary surgery. Patients in remission with minimal disease burdens are ideal candidates for anti-tumor immune augmentation strategies aimed at cure or prolonging disease-free periods. However, immunosuppressive pathways in the tumor microenvironment are obstacles to durable antitumor immunity. There is thus an ongoing and unmet need for improved treatment modalities for EOC, as well as other cancers

### DESCRIPTION OF FIGURES

FIG. 1. Peritoneal macrophages from non-tumor-bearing and IDB-MOSEC-bearing mice suppress T cell proliferation. A) Macrophages are the predominant peritoneal myeloid cell in naive and IDB-MOSEC-bearing mice. Representative dot-plots showing peritoneal macrophages (CD11b⁺/F4/80⁺), granulocytic MDSCs (CD11b⁺Ly6G⁺Ly6C^{low}), and monocytic MDSCs (CD11b⁺Ly6C⁺Ly6G⁻) in non-tumor-bearing and MOSEC-bearing mice (1 mouse/group) at day 42 and 90. CD11b⁺ populations from total cells were gated to obtain percent of macrophages, granulocytic and monocytic MDSCs. B) Resident peritoneal macrophages abrogate anti-CD3/B7.1-stimulated CD4⁺ and CD8⁺ T cell proliferation. Column-purified myeloid (>95% CD11b⁺F4/80⁺, Mphages) PECs from non-tumor-bearing mice (n=6) were co-cultured with CFSE-labeled splenocytes from naive mice (E:T ratio: 1:1) in triplicate in anti-CD3/B7.1-coated 96-well plates. After 72 hours of culture, CD4⁺ and CD8⁺ T cell proliferation was assessed based on CFSE dilution as described in methods. Data are representative of 3 independent experiments. C) Resident peritoneal macrophages mediate suppression of CD4⁺ and CD8⁺ T cell proliferation in a cell-cell contact-dependent manner. Similar to B, purified myeloid (>95% CD11b⁺F4/80⁺) cells from non-tumor-bearing mice (n=20) and CFSE-labeled splenocytes were co-cultured in a 24-well plate transwell system, and after 72 hours T cell proliferation was assessed as described in methods. Data are representative of 2 independent experiments. D) Peritoneal macrophages from ovarian tumor-bearing mice suppress T cell proliferation independent of NADPH oxidase. Column-purified peritoneal macrophages (CD11b⁺F4/80⁺) from wild-type (WT-Mphage) and NADPH oxidase-deficient (p47^{phox-/-}-Mphage) mice at day 90 after ID8-MOSEC administration were evaluated for their effects on anti-CD3/B7.1 stimulated CD4⁺ and CD8⁺ T cell suppression. Data are representative of 3 mice per group from 3 separate experiments.

### FIG. 2. Vaccination with MIS416 Increases Accumulation of OT-I Cells in the Tumor Microenvironment and Systemically in Ovarian Tumor-Bearing Mice

At day 30 after i.p. IE9-MOSEC administration, mice were adoptively transferred with OT-I cells, followed by immunization with MIS416 mixed with OVA (days 31 and 38) or vehicle mixed with OVA (control). Lymph node cells (LN), splenocytes (Spl) and PECs harvested at day 43 and 59 after tumor administration were analyzed for OT-I cell accumulation (n=3 mice per group per time point). A and B) MIS416 administration increased accumulation of OT-I cells in the local peritoneal environment and systemically at day 43 after tumor challenge. A) Representative dot plots of MIS614 and vehicle groups showing accumulation of CD8⁺Thy1.1⁺ (%) cells in different compartments. Total cells were gated to obtain the percent positive cells for CD8 and Thy1.1. B) On day 59 after tumor administration, OT-I cell accumulation had substantially waned in MIS416-treated mice. Data are representative of 3 mice per group from 2 separate experiments. C) The percent of interferon γ-producing and granzyme B-expresing (IFNγ⁺CD107α⁺ and GrB⁺CD107α⁺) peritoneal OT-I cells in MIS416 vaccinated and control IE9-MOSEC-bearing mice (n=3 mice/group) at day 59 after tumor implant was analyzed by intracellular staining of PECs. The proportion of peritoneal OT-I cells expressing granzyme B, CD107α (a marker for degranulation), and dual expression of these markers was similar in MIS416-treated compared to control mice, and the proportion expressing interferon-γ was ≦2% in both groups (FIG. 2C). These results show that MIS416 dramatically increases the accumulation of OT-I cells both in the local tumor microenvironment and systemically in tumor-bearing mice, without significantly altering the effector phenotypes of these cells. However, the effect on OT-I cell expansion was short-lived, and the overall effect of vaccination on time to euthanasia was modest.

FIG. 3. MIS416 vaccination increases the accumulation of immunosuppressive myeloid cells in the peritoneum of ovarian tumor bearing mice. At day 30 after i.p. IE9-MOSEC administration, mice were adoptively transferred with OT-I cells, followed by immunization with MIS416 mixed with OVA (days 31 and 38) or vehicle mixed with OVA (control). A and B) MIS416 administration led to an increased accumulation of total myeloid cells (CD11b⁺) and of multiple myeloid subsets in the peritoneum at day 43 after tumor administration. A) Representative dot plots of a single mouse from each group show substantial increase in the total myeloid (CD11b⁺) cells in PECs from MIS416 vaccinated mice compared to controls. B) Accumulation of myeloid cells (CD11b⁺) and specific myeloid subsets: DCs (CD11b⁺CD11c⁺), macrophages (CD11b⁺F4/80⁺), granulocytic (CD11b⁺Ly6G⁺) and monocytic (CD11b⁺Ly6C⁺) cells in PECs isolated from MIS416 vaccinated and PBS-treated mice at day 43. C) MIS416 vaccination led to increased accumulation of granulocytic MDSCs)(CD11b⁺Ly6G⁺Ly6C^{lo}) in the local tumor microenvironment and systemically on day 43. By day 59, no significant difference between MIS416 and PBS groups was observed. D and E) Cytological analysis of PECs showed increased granulocytic cell accumulation in MIS416 vs. PBS-treated mice on day 43. In both groups, macrophages predominated. Granulocytic cells were rarely observed in PBS-treated mice (D) and were more prominent in MIS416-treated mice (E) White arrow, tumor cells; black arrows, granulocytic cells. F) The proportion of peritoneal macrophages expressing M2 markers, CD206 and IL-4R, increased in tumor-bearing mice, but was not consistently affected by MIS416 administration (NTB, non-tumor-bearing). G) Peritoneal myeloid cells from MIS416 and PBS-treated tumor-bearing mice suppressed T cell proliferation. Column-purified CD11b PECs harvested at day 59 after IE9-MOSEC implant (corresponding to 21 days after vaccination) were assessed for suppression of anti-CD3/B7.1-stimulated CD4 and CD8 T cell proliferation. Non-myeloid (CD11b⁻) PECs from both groups of mice, which predominantly contained tumor cells and lymphocytes, were not suppressive. Data are representative of 3 mice per group from 3 separate experiments.

FIG. 4. Myeloid cell depletion enhances the efficacy of MIS416 immunization in MOSEC-bearing mice. A) Adoptive transfer (AT) of OT-I cells was performed in IE9-MOSEC-bearing mice at day 30. Vaccination (MIS416 plus OVA) or control (vehicle plus OVA) treatments were administered on days 31 and 38. One week after the second immunization, mice were administered weekly anti-CD11b mAb or control IgG (isotype) for 6 weeks, and monitored for tumor progression requiring euthanasia. Time to euthanasia was displayed by Kaplan-Meier plots (n=16 mice per group). MIS416 vaccination followed by isotype significantly prolonged time to euthanasia compared to treatment with vehicle followed by isotype (log-rank, p<0.0001). MIS416 vaccination followed by anti-CD11b mAb treatment led to significantly prolonged survival compared to MIS416 vaccination followed by isotype (p<0.0013). In the absence of prior MIS416 vaccination, anti-CD11b had no benefit. B) Similarly treated mice (n=3 per treatment group) were pre-selected for sacrifice at day 59 after tumor challenge, and visible tumor was removed and weighed. MIS416-treated mice had reduced tumor weight compared with non-MIS416 groups (p=0.004; adjusted p-value for multiple comparisons=0.01), while anti-CD11b had no significant effect.

FIG. 5. Heterogeneity in ascitic myeloid cell populations and immunosuppressive activity in patients with advanced EOC. Myeloid cells from ascites collected at the time of primary surgery from 8 patients with EOC were evaluated. A) Gating on CD33high (gate 4) and CD33medium (gate 3) myeloid populations, the proportion of macrophages (DR+CD15−), granulocytic (DR-CD15+), myelomonocytic (DR+CD15+), and immature myeloid (DR-CD15−) cells was determined B and C) There were dramatic differences in the composition and immunosuppressive phenotype of ascitic myeloid cells. As examples, while the peritoneal myeloid fraction of patient 1 (B) contained a mixed population of mature macrophages, granulocytic cells, mixed myelomonocytic cells and immature cells, a paucity of granulocytic cells was present in the ascites of patient 2 (C). Cytology of ascites from these patients was consistent with the flow cytometry data (black arrows, granulocytic cells;

grey arrow, tumor cell; white arrow, collection of macrophages). Both mature macrophages and non-macrophage myeloid cells (MDSC-rich fraction) from patient 1 suppressed stimulated T cell proliferation to basal levels while peritoneal macrophages from patient 2 did not suppress stimulated T cell proliferation. Non-myeloid PECs from all samples had modest or no T cell suppressive activity.

FIG. 6. Anti-CD11b mAb treatment partially depletes peritoneal myeloid cells. Mice were administered i.p. IE9-MOSEC, followed by administration of anti-CD11b mAb or isotype on days 50 and 57 and sacrifice on day 59. Representative dot plots of PECs show that anti-CD11b treatment partially depleted all myeloid subsets analyzed, with the major effect on peritoneal granulocytic MDSCs (CD11b+Ly6G+Ly6Clow). Data are representative of 3 mice per group.

### SUMMARY

This present disclosure comprises a method for enhancing the effect of an anti-cancer agent. The method comprises concurrently or sequentially administering to an individual who is in need of therapy for a tumor i) the anti-cancer agent; and ii) an agent that causes depletion of myeloid cells and/or inhibits recruitment of myeloid cells to the tumor, wherein the effect of the anti-cancer agent on the tumor is greater relative to the effect of the anti-cancer agent in the absence of the agent that causes depletion of myeloid cells and/or inhibits recruitment of myeloid cells to the tumor. In embodiments, the myeloid cells are macrophages or granulocytes, or a combination thereof. In one embodiment, the individual is in need of therapy for an ovarian tumor. In certain approaches, the agent that causes depletion of myeloid cells and/or inhibits recruitment of myeloid cells to the tumor is a monoclonal antibody (mAb) that specifically recognizes the myeloid cells. In certain embodiments, the mAb is selected from an anti-CD11b mAb and an anti-CD33 mAb. In an embodiment, the anti-cancer agent is an anti-tumor vaccine. In an embodiment the anti-cancer agent is MIS416.

In another aspect the disclosure include a method for determining whether or not an individual is a candidate for a tumor therapy comprising i) an anti-cancer agent and ii) an agent that causes depletion of myeloid cells and/or inhibits recruitment of myeloid cells to the tumor, the method comprising determining whether the individual has a tumor characterized by undesirable myeloid cell proliferation and/or tumor infiltration and/or myeloid cell recruitment to the tumor, and if such determination is made, designating the individual as a candidate for the therapy. In one embodiment the method further comprises administering to the individual the anti-cancer agent and the agent that causes depletion of myeloid cells and/or inhibits recruitment of myeloid cells to the tumor.

### DETAILED DESCRIPTION OF THE INVENTION

The present disclosure provides approaches for enhancing the efficacy of vaccines. In general, the method comprises vaccinating an individual against cancer, and subsequent to the vaccination, depleting myeloid cells in the individual, such as in a tumor microenvironment. In developing the present disclosure, we demonstrate using an orthotopic syngeneic mouse model of epithelial ovarian cancer, that immunosuppressive macrophages and myeloid-derived suppressor cells (MDSCs) accumulate in the local tumor environment, correlating with disease burden. In addition, resident peritoneal macrophages from non-tumor-bearing mice were highly immunosuppressive, abrogating stimulated T cell proliferation in a cell contact-dependent fashion Immunization with stimulatory microparticles comprising TLR9 and NOD-2 ligands (MIS416) significantly prolonged survival in tumor-bearing mice. The strategy of MIS416 immunization followed by anti-CD11b myeloid cell depletion further delayed tumor progression, thereby establishing that myeloid depletion can enhance vaccine efficacy.

In more detail, our overall hypothesis is that anti-tumor vaccine efficacy would be enhanced if followed by myeloid cell depletion. MIS416 is a novel immune adjuvant microparticle comprised of immune-stimulatory muramyl dipeptide and bacterial DNA, and is capable of inducing DC maturation and cross-presentation that promotes CTL polarization and Th1 immunity. As little is currently known about its role as a cancer vaccine adjuvant and a modulator of the tumor microenvironment, we investigated MIS416 in an orthotopic syngeneic murine model of EOC, which is a clinically relevant mouse model. Since the tumor cell line used does not express known unique antigens, it was engineered to express ovalbumin (OVA) as a nominal tumor antigen Immunization with MIS416 plus OVA modestly delayed tumor progression, but was also associated with increased peritoneal accumulation of granulocytic MDSCs, which are predicted to impede durable anti-tumor immunity. Although CD11b+ myeloid cell depletion by itself had no benefit, sequential immunization followed by myeloid cell depletion led to significant delay in tumor progression compared to vaccination alone. Thus, we demonstrate that the combination of vaccination and myeloid cell depletion is superior to using either approach alone. As such, it is expected that the invention is suitable for use with any cancer, wherein a presence of undesirable myeloid cells is considered to aggravate the cancer condition and/or is positively correlated with infiltration and/or proliferation of such myeloid cells. The invention is thus applicable to ovarian cancer. Furthermore, since immunosuppressive myeloid populations can accumulate systemically and in the local microenvironment of numerous tumors (e.g., breast, melanoma, renal, lung), this invention, in principle, is applicable to all cancers.

Without intending to be limited to any particular theory, it is considered that the description and data presented herein show that the EOC tumor microenvironment is characterized by accumulation of immunosuppressive myeloid cells and that that this condition inhibits and/or abrogates vaccine-induced immunity. Thus, in various embodiments, the invention provides for immunization at an early stage of disease, and subsequent to the immunization, depleting myeloid cells and/or inhibiting their accumulation in the tumor microenvironment. Myeloid cell populations targeted by this invention include but are not necessarily limited to macrophages and myeloid-derived suppressor cells (MDSCs). MDSCs are heterogeneous cell populations that are defined by their myeloid origin, immature state and ability to potently suppress T cell responses.

In one embodiment, the method of the invention comprises sequentially: i) administering an anti-cancer agent to an individual; and ii) administering an agent that causes depletion and/or inhibition of recruitment of myeloid cells, such as in or near the tumor microenvironment. For ease of reference the anti-myeloid cell agent will be referred to herein from time to time as an anti-MCA.

It is expected that the invention can be used in connection with any anti-cancer agent, because irrespective of the anti-cancer agent, the premise of reducing myeloid cells which are thought to be interfering with the activity and/or access of therapeutic agents to the tumor remains the same. Immune-based therapies can have both desirable (e.g., promoting tumor regression) and undesirable (e.g., promoting tumor progression) effects in shaping the immune response to tumors. In certain embodiments, although vaccination can have the net effect of delaying tumor progression, it may also induce immune responses that at least partially limit the beneficial effects of vaccination. In one embodiment the agent is referred to as MIS- 416 or MIS416. This agent is a combination of microparticles and naturally occurring, non-immunogenic, cytosolically-active TLR-9 and NOD-2 ligands and is produced by, for example, Innate Immunotherapeutics Limited, New Zealand. Although administration of MIS416 delays tumor progression, it has the undesirable effect of promoting accumulation of peritoneal myeloid cells, including MDSCs, which can abrogate durable anti-tumor immunity.

The present invention addresses these undesirable effects at least in part by providing a method comprising sequential administration of MIS416 (vaccination) followed by anti-CD11b, which depletes and/or inhibits recruitment of myeloid cells in the peritoneal tumor microenvironment.

The agent that is intended to reduce the myeloid cells can be any agent that can selectively recognize such cells, and directly or indirectly deplete their numbers and/or inhibit their recruitment to the tumor microenvironment. Preferred agents include those that specifically recognize any pan-myeloid marker. As such, the anti-MCA can be a mAb that can specifically recognize myeloid cells, such as by specifically binding to any myeloid cell surface marker. In certain embodiments, the anti-MCA can be used to deplete myeloid cells by specifically binding to them, resulting in their selective removal/destruction via the endogenous activity of the immune system of the individual to whom the anti-MCA is administered. In other embodiments, the anti-MCA can be conjugated to a cytotoxic moiety, such that targeted delivery of the anti-MCA results in death of the cells via at least in part by activity of the cytotoxic agent. In another embodiment, this invention may impede myeloid cell trafficking from the circulation to the tumor microenvironment. In an embodiment, the anti-MCA is a mAb that specifically recognizes either component, or a complex of Mac-1, which is composed of CD11b and CD18.

Thus, the invention includes any myeloid depletion strategy or strategy to suppress myeloid cell recruitment using any anti-MCA. In one embodiment, the method entails myeloid cell depletion in the peritoneum of an individual.

In one embodiment, the activity of a vaccine is improved by administering an anti-CD11b mAb as the anti-MCA, or an anti-CD33 mAb. Anti-CD11b mAb is commercially available and has been tested in humans for numerous diseases, has been shown to be safe, but has not been shown to alone be therapeutically effective for non-malignant diseases. However, we demonstrate its utility in the present invention, which also demonstrates the feasibility of the anti-MCA approach generally.

Anti-MCA agents, such as mAbs directed against myeloid cell surface markers, can be administered using any suitable technique, such as intravenous injection, intra-tumor injection, or peritoneal administration. Dosing will depend on factors known to those of skill in the art, including but not limited to the type of cancer being treated, the age, gender and overall health of the individual patient, and the stage of the disease. The optimal timing and number of anti-MCA administrations can be determined using conventional techniques, given the benefit of the present invention.

In another aspect, the invention comprises identifying an individual as a candidate for a therapy provided by the invention. In general, this aspect comprises testing the individual for cancer that is characterized at least in part by undesirable myeloid cell accumulation and/or tumor infiltration, and if such undesirable myeloid cell proliferation and/or tumor infiltration is identified, prescribing and/or administering to the individual a composition comprising a vaccine directed against the cancer, and subsequent to vaccination, administering an anti-MCA agent and described herein. The invention also includes combining these approaches with surgical interventions, including but not necessarily limited to debulking malignant and/or peripheral tissue.

The following specific examples are provided to illustrate the invention, but are not intended to be limiting in any way.

### EXAMPLE 1

This Example demonstrates that resident and tumor-associated peritoneal macrophages in mice suppress T cell proliferation.

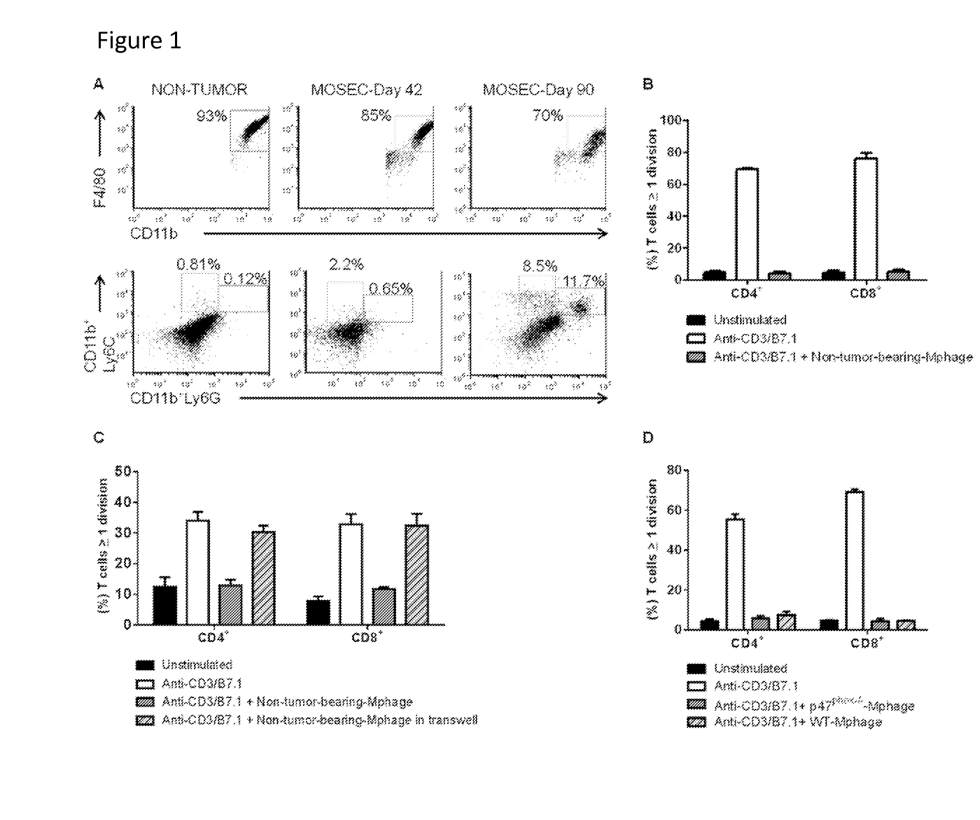

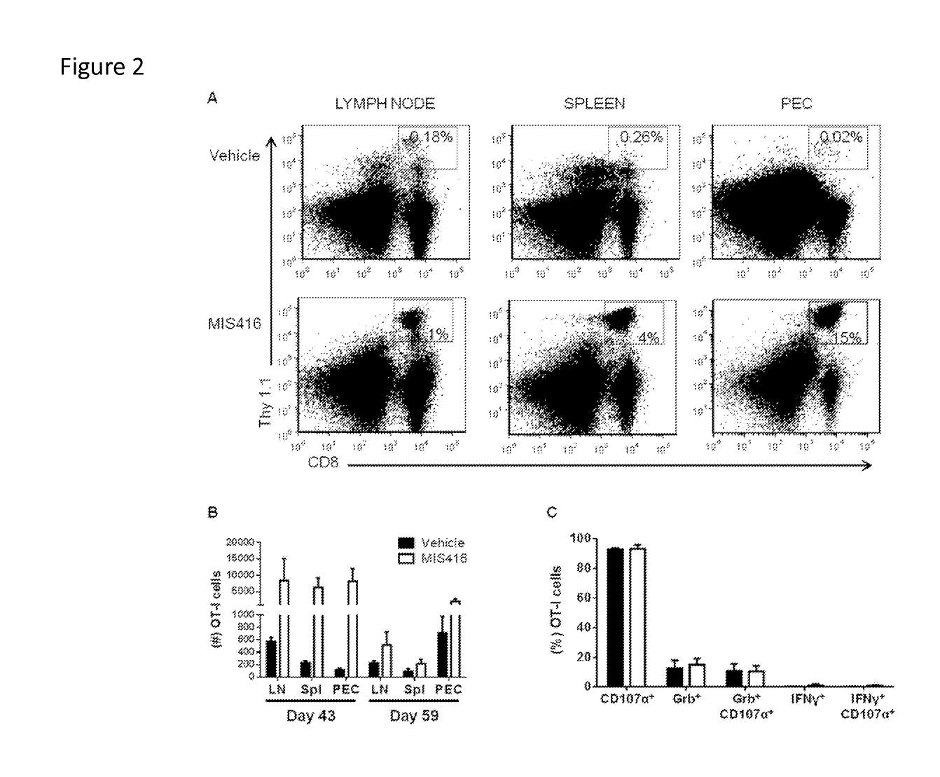

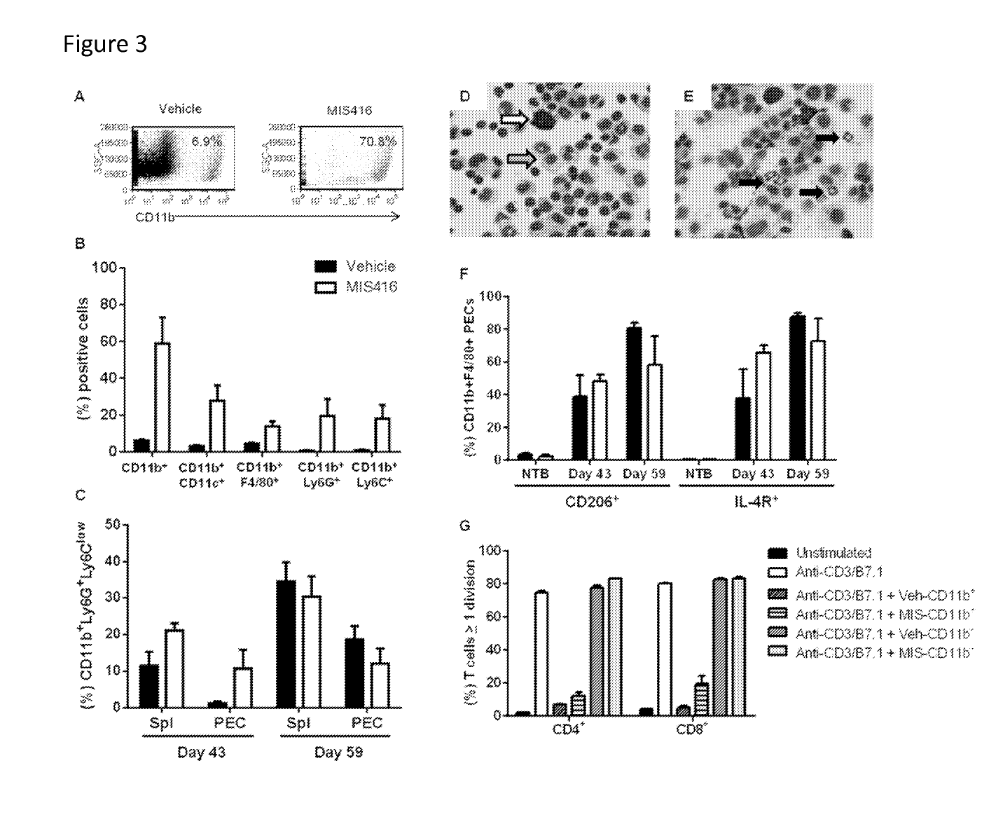

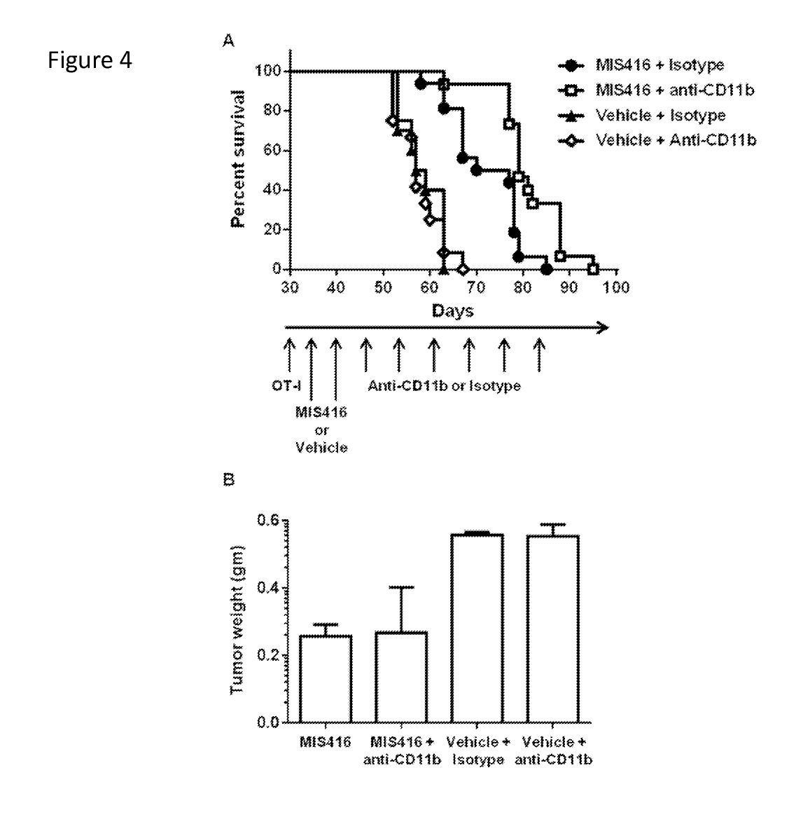

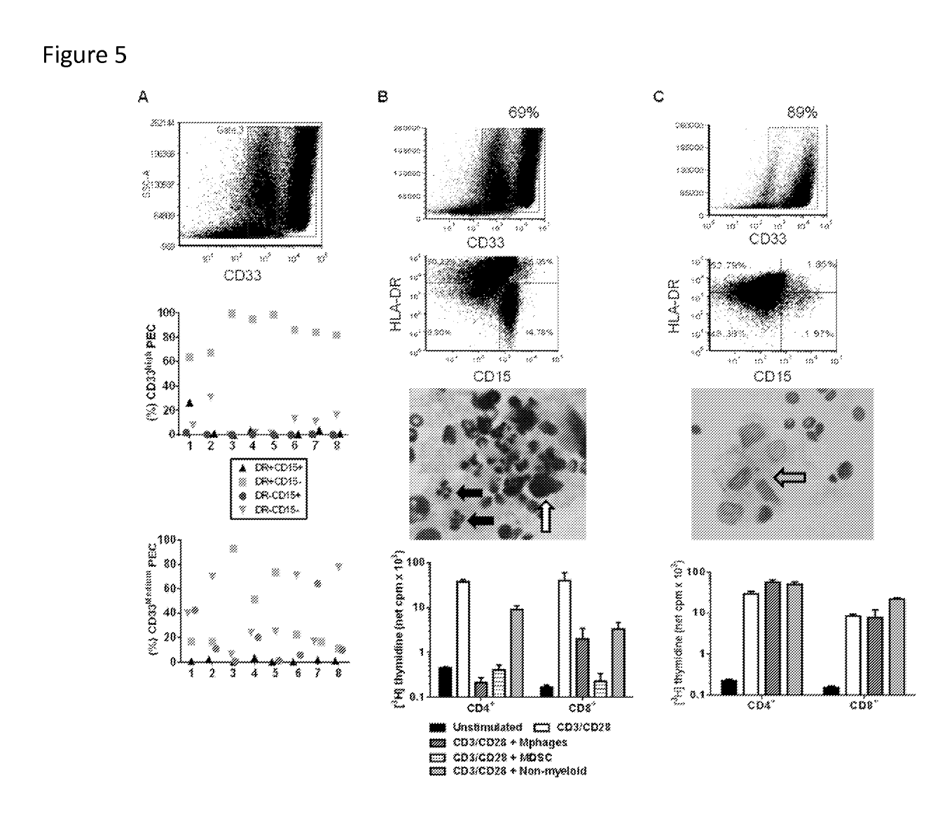

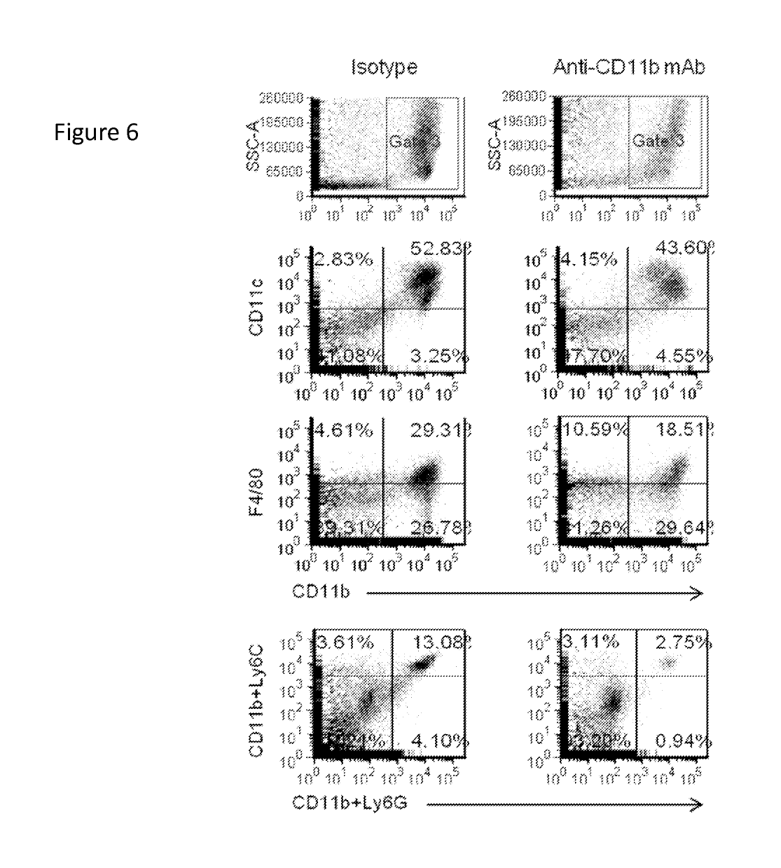

We observed that in syngeneic murine EOC (MOSEC), peritoneal granulocytic MDSCs (CD11b⁺Ly6G⁺Ly6C^{low}) accumulated in the peritoneum as a function of tumor burden, and suppressed stimulated T cell proliferation, while non-myeloid (CD11b−) peritoneal cells from tumor-bearing mice either incompletely suppressed or had no effect on stimulated T cell proliferation ex vivo. We evaluated the effects of peritoneal macrophages from both non-tumor and tumor-bearing mice on stimulated T cell proliferation. In non-tumor-bearing naive mice, peritoneal myeloid cells were >90% macrophages (CD11b⁺F4/80⁺) (FIG. 1A). In syngeneic murine EOC (MOSEC), macrophages constituted the predominant population of peritoneal myeloid cells, with variable numbers of granulocytic MDSCs and monocytic MDSCs (CD11b⁺Ly6C⁺Ly6G⁻) detected at both early (day 42 after tumor challenge) and advanced (day 90) disease stages (FIG. 1A). Purified resident peritoneal macrophages from naive mice abrogated anti-CD3/B7.1-stimulated CD4⁺ and CD8⁺ T cell proliferation ex-vivo (FIG. 1B), substantiating that the ovarian environment is inherently immunosuppressive. Similarly almost complete inhibition of T cell proliferation was also associated with peritoneal macrophages (CD11b⁺F4/80⁺) isolated from MOSEC mice at day 90 after tumor challenge (FIG. 1D; WT Mphage). We next evaluated whether resident macrophage-mediated T cell suppression was contact-dependent using the transwell system, and found that the absence of cell-cell contact abrogated the suppressive effect of peritoneal macrophages from unstimulated mice (FIG. 1C). Since reactive oxidant intermediates produced by myeloid cells can have important intracellular and intercellular signaling functions, we also evaluated whether NADPH oxidase in macrophages was relevant to their T cell suppressive function. Peritoneal macrophages from MOSEC-bearing NADPH oxidase-deficient (p47^{phox-/-}-Mphage) mice suppressed ex vivo T cell proliferation to a similar degree as MOSEC WT macrophages (FIG. 1D). These observations demonstrate both resident and tumor-associated peritoneal macrophages may contribute to the immunosuppressive milieu in the EOC tumor microenvironment, which may be a barrier to anti-tumor immunity.

### EXAMPLE 2

This Example demonstrates that MIS416 vaccination augments antigen-specific CTL expansion, but promotes accumulation of granulocytic MDSCs in murine EOC.

We next evaluated whether vaccination could mitigate the immunosuppressive environment in EOC and prolong survival. Mice were administered i.p. OVA-expressing MOSEC (IE9 cells; 1×10⁷ cells/mouse). At a time point corresponding to low disease burden (day 30) mice were adoptively transferred with OT-I cells, followed by immunization with MIS416 mixed with OVA (days 31 and 38). In this model, we are able to track tumor progression and T cell responses to OVA, which is being used as a tumor-associated antigen. This approach is necessary, since there are no well-defined endogenous unique tumor antigens in this syngeneic tumor model. In two separate experiments, MIS416 immunization extended the median time to tumor progression requiring euthanasia by approximately 2 weeks (see FIG. 4).

Since MIS416 was modestly protective, we next determined its effects on CTL and myeloid responses to understand mechanisms for the lack of more durable anti-tumor responses. Tumor-bearing mice treated with MIS416 or vehicle on days 31 and 38 (n=3 per treatment group per time point) were sacrificed on days 43 or 59 in relation to 1E9 administration, corresponding to early and more advanced tumor burden, respectively. At day 43, MIS416 administration led to a dramatic increase in OT-I cell accumulation in PEC, TDLN, and spleens (FIG. 2A). However, at day 59, OT-I cell accumulation had substantially waned in MIS416-treated mice (FIG. 2B). The proportion of peritoneal OT-I cells expressing granzyme B, CD107a (a marker for degranulation), and dual expression of these markers was similar in MIS416-treated compared to control mice, and the proportion expressing interferon-γ was ≦2% in both groups (FIG. 2C). These results show that MIS416 dramatically increases the accumulation of OT-I cells both in the local tumor microenvironment and systemically in tumor-bearing mice, without significantly altering the effector phenotypes of these cells. However, the effect on OT-I cell expansion was short-lived, and the overall effect of vaccination on time to euthanasia was modest.

Since EOC progression is associated with the accumulation of immunosuppressive myeloid cells, we next evaluated the effect of MIS416 on local and systemic myeloid cell accumulation and immunosuppressive phenotype in tumor-bearing mice. MIS416 administration led to an increased accumulation of total myeloid cells (CD11b+) consisting of multiple myeloid subsets in the peritoneum at day 43 after tumor administration (FIGS. 3A and B). MIS416-treated mice had increased accumulation of granulocytic MDSCs (CD11b+Ly6G+Ly6Clow) in the local tumor microenvironment and in spleens on day 43 (FIG. 3C). By day 59, no significant difference between MIS416 and vehicle groups was observed. Cytology of unfractionated PECs confirmed the accumulation of cells with granulocytic morphology in MIS416-vaccinated mice, while these cells were virtually absent in control mice (FIGS. 3D and E). Further macrophage (CD11b+F4/80+) subset analysis for the M2 markers CD206 and IL-4R showed in non-tumor-bearing mice only a small percentage (≦5%) of peritoneal macrophages expressed M2 markers while, in contrast, the majority expressed M2 markers at day 59 after tumor administration (FIG. 3F). We did not observe a consistent effect of MIS416 on the proportion of peritoneal macrophages expressing M2 markers in tumor-bearing mice (FIG. 3F). Peritoneal myeloid cells (CD11b+) from both MIS416− and vehicle-treated mice abrogated stimulated T cell proliferation while the non-myeloid fraction had no significant effect (FIG. 3G). These results show that an early effect of MIS416 vaccination is to increase the accumulation of myeloid cells, including granulocytic MDSCs, in the local tumor microenvironment (day 43), while at a later time point (day 59) corresponding to more advanced tumor burden, this effect of MIS416 was no longer detectable.

### EXAMPLE 3

This Example demonstrates that myeloid cell depletion enhances MIS416 vaccine efficacy against ovarian tumor.

Since MIS416 vaccination enhanced antigen-specific CTL accumulation in the tumor microenvironment and systemically while also promoting the accumulation of immunosuppressive myeloid cells, we reasoned that vaccination followed by non-selective myeloid depletion using anti-CD11b mAb to target both tumor-associated macrophages and MDSCs, may prolong vaccine-induced anti-tumor immunity. The primary endpoint was time to euthanasia based on pre-specified morbidity criteria. Anti-CD11b mAb treatment (or isotype) was begun 3 weeks after adoptive transfer of OT-I cells and the initial MIS416 vaccination allowing sufficient time to induce expansion of transferred OT-I cells (FIG. 2). Anti-CD11b administration led to depletion of all subsets of myeloid cells (macrophage, DC and MDSC), with a >4-fold depletion of granulocytic MDSCs (FIG. 6). MIS416 vaccination alone significantly increased time to tumor progression requiring euthanasia (p<0.0001) (FIG. 4A). Although anti-CD11b mAb by itself had no effect on tumor progression, the strategy of MIS416 vaccination followed by anti-CD11b significantly prolonged survival compared to vaccination followed by isotype (p=0.0013) (FIG. 4A). Mice pre-selected for sacrifice at day 59 after tumor challenge (n=3 per treatment group) confirmed there was reduced tumor weight in MIS416-treated versus non-MIS416-treated mice, but there was no effect of anti-CD11b treatment (FIG. 4B). Together these data show that: (i) MOSEC tumor growth leads to an accumulation of immunosuppressive macrophages and MDSCs in the peritoneal tumor microenvironment; (ii) MIS416 vaccination prolongs survival of tumor-bearing mice, and modulates both host myeloid cells and adoptively transferred OT-I cell accumulation; (iii) Sequential vaccination followed by myeloid cell depletion significantly extends time to tumor progression requiring euthanasia.

### EXAMPLE 4

This Example demonstrates heterogeneity in ascitic myeloid cell accumulation and immunosuppressive phenotype in patients with advanced EOC.

Based on our data from murine EOC, we undertook a more detailed analysis of macrophages and MDSCs in ascites of patients with EOC and evaluated their functional properties. Myeloid cells from ascites collected at the time of primary surgery from 8 patients were evaluated. Macrophages were defined based on CD33+DR+CD15− expression, granulocytic cells were defined based on CD33+DR−CD15+ expression, a mixed myelomonocytic lineage was defined by CD33+DR+CD15+ expression, and immature myeloid cells were defined based on lack of expression of macrophage or granulocytic markers (CD33+DR−CD15−). CD33medium and CD33high populations were observed in all patients, with the proportion of each population varying among patients. Mature macrophages principally segregated in the CD33high group, virtually all granulocytic cells were CD33medium, and immature myeloid cells were observed in both CD33medium and CD33high groups. There was substantial inter-patient variability in the proportion of myeloid cell populations in ascites (FIG. 5A). This variability was most obvious in the granulocytic cell population, which made up a significant population of myeloid cells in the ascites of some patients and was virtually absent in others.

We next evaluated the immunosuppressive function of ascitic macrophages and MDSCs. Since MDSCs are a heterogeneous population of immature cells, and expression of surface markers can overlap with mature myeloid cells, we applied stringent criteria to FACS purification of macrophages, requiring high surface expression of 2 markers (CD14 and DR) expressed at late stages of macrophage differentiation. The remaining myeloid cell population was defined as MDSCs if they exhibited immunosuppressive function. The immunosuppressive function of myeloid PECs in ascites was defined based on their ability to suppress proliferation of purified anti-CD3/CD28-stimulated allogeneic T cells from a normal volunteer, as described (Solito S, Falisi E, Diaz-Montero CM, Doni A, Pinton L, Rosato A, et al. A human promyelocytic-like population is responsible for the immune suppression mediated by myeloid-derived suppressor cells. Blood. 2011;118:2254-65.). All T cells were purified from the same normal donor, and processed using a standard protocol. We found striking inter-patient variability in the immunosuppressive properties of myeloid PECs from the 8 patients tested. As illustrated in FIG. 5B, both mature macrophages and non-macrophage myeloid cells (MDSC-rich fraction) from patient 1 suppressed stimulated T cell proliferation to basal levels. In contrast, peritoneal macrophages from patient 2 did not suppress stimulated

T cell proliferation (FIG. 5C). The sort-purified MDSC population from patient 2 contained insufficient cell numbers to include in this experiment. Non-myeloid PECs from all samples had modest or no T cell suppressive activity. Together, these results raise the potential for distinct populations of ascites myeloid cells that can suppress T cell immunity in the tumor microenvironment in patients with advanced EOC.

It will be apparent to those skilled in the art from the foregoing that our results in murine models and in patients with EOC show that that immunosuppressive MDSCs accumulate in the local tumor environment and also systemically as a function of disease burden, suppressing T cell immunity, which likely facilitates tumor progression. Immunization with MIS416 significantly prolonged survival in tumor-bearing mice, but was also associated with increased local and systemic accumulation of granulocytic MDSCs. The strategy of vaccination followed by broad myeloid cell depletion using anti-CD11b mAb significantly delayed tumor progression compared to vaccination alone, demonstrating that myeloid depletion can enhance vaccine efficacy. Even with incomplete myeloid cell depletion, we observed a modest but statistically significant effect in enhancing vaccine efficacy.

In summary, results presented in this disclosure indicate that peritoneal macrophages contribute to a locally immunosuppressive environment in the absence of tumor, and innate immune responses during EOC further abrogate cellular immunity. Consistent with this, myeloid cell depletion enhanced anti-tumor vaccine efficacy in murine EOC. In humans,

EOC leads to an accumulation of a peritoneal myeloid cell population consisting of mature macrophages, immature myeloid cells and granulocytic cells with variable immunosuppressive phenotypes. Furthermore, the strong correlation between the enhancement of immunosuppressive macrophages and MDSCs in the tumor microenvironment and disease progression suggests that delineation of the myeloid inflammatory composition and immunosuppressive function would be of prognostic significance.

### EXAMPLE 5

The following materials and methods were used to obtain the data presented in the foregoing Examples.

Mice: Female C57BL/6, OVA-specific TCR transgenic OT-I/Rag^{-/-} mice (Jackson Laboratory, Bar Harbor, Me.), and NADPH oxidase-deficient (p47^{phox-/-} ) mice (13) were used at 6-8 weeks of age. All mice were maintained under specific pathogen free conditions at the animal care facility at Roswell Park Cancer Institute (RPCI) and used in compliance with all relevant laws and institutional guidelines under a protocol approved by the RPCI Animal Care and Use Committee.

Mouse ovarian surface epithelial cancer (MOSEC) cells: The IDB MOSEC line (provided by Dr. P. Terranova, University of Kansas Medical Center, Kansas City, Kans.) was derived from epithelial ovarian cells harvested from female C57BL/6 mice that were passaged in vitro. Intraperitoneal (i.p.) injection of clonal lines established from late passage epithelial cells from syngeneic tumors in mice results in ascites and peritoneal implants that mimic the human disease (Godoy et a. PloS one. 2013; 8:e69631, Roby et al. Carcinogenesis. 2000; 21:585-91). The OVA-expressing 1E9 cell line was generated as described (Tomihara et al. J Immunol. 2010; 184:6151-60). IDB and 1E9 MOSEC cells were cultured in RPMI 1640 media with heat-inactivated FBS (10%), L-glutamine (2 mM), HEPES (25 mM), sodium pyruvate (1 mM), 2-mercaptoethanol (50 μM), penicillin/streptomycin (100 μg/ml) and non-essential amino acids.

Tumor administration: Mice were administered i.p. ID8 or 1E9 cells (5-10×10⁶ cells in PBS), and were monitored daily for 100 days by trained animal care staff blinded to treatment regimens. Moribund mice were euthanized based on the decisions of animal care staff using pre-specified criteria (abdominal distention, lethargy or inability to ambulate). Pre-selected groups of tumor-bearing mice were sacrificed prior to the onset of morbidity for immunologic endpoints.

Adoptive transfer of OT-I cells: On day 30 after 1E9 administration, all tumor-bearing mice underwent adoptive transfer of OT-I lymphocytes (3×10⁶ cells/0.2 ml PBS/mouse) by retro-orbital injection. Lymph nodes (inguinal, popliteal, brachial, axillary, maxillary, periaortic, and mesenteric) harvested from OT-I mice were homogenized in sterile conditions. Single lymph node cell suspension was prepared in PBS and purity of OT-I cells (>90%) was confirmed by flow cytometry with anti-CD8 and anti-Thy 1.1 mAb prior to injection.

Generation of anti-CD11b mAb: Anti-CD1lb mAb was generated from ascites of SCID mice after i.p. administration of M1/70 hybridoma (DSHB, University of Iowa, Iowa City, Iowa) in the Laboratory Animal Research facility at RPCI. The ascites was heat-inactivated and filter-sterilized before in vivo administration. In vivo titration experiments were conducted in non-tumor-bearing and MOSEC-bearing mice using different volumes of ascites (25-200 μl) to measure depletion of CD11b⁺ cells (macrophages, myeloid DCs and neutrophils) in the peritoneum and spleen. Based on >70% depletion of myeloid cells, anti-CD11b mAb (50 μl) was selected for therapeutic depletion studies. Depletion of myeloid cells was confirmed in the tumor microenvironment by flow cytometry.

MIS416 vaccination and anti-CD11b treatment: Mice were assigned into 4 groups: 1) MIS416 and anti-CD11b; 2) PBS and anti-CD11b; 3) MIS416 and IgG isotype; 4) PBS and IgG isotype. MIS416 (5.5 mg/ml) or PBS was mixed with OVA solution (180 □g/ml) at 1:1 ratio, and 200 μl per mouse was administered subcutaneously on days 31 and 38 in relation to tumor administration. Beginning on day 52, mice were treated with i.p. anti-CD11b mAb (50 μl ascitic fluid in 150 μl PBS/mouse) or isotype IgG weekly for 6 weeks or until sacrificed.

Immunological analysis in mice: Following sacrifice of mice, peritoneal exudate cells (PECs) were collected by peritoneal lavage with PBS (5-8 ml, containing 1% FBS and 0.5 mM EDTA). PECs were subjected to RBC lysis with ACK buffer, followed by washing. Tumor-draining lymph nodes (TDLN) and spleens were also collected at harvest, and single cell suspensions were subjected to RBC lysis with ACK buffer, followed by washing. Isolated PECs, splenocytes, and TDLN cells were either used within 24 h of harvest for flow cytometry and functional studies or frozen in liquid nitrogen in media containing 20% FBS and 5% DMSO. To evaluate cellular morphology, PECs from each group of mice were analyzed microscopically by Diff-Quick-stained cytospins (Fisher Scientific, Kalamazoo, Mich.).

Flow cytometry analysis was conducted on a FACScan (Becton Dickinson, Franklin Lakes, N.J.). Forward scatter versus side scatter gating was set to include all non-aggregated cells. Data were analyzed using FCS Express 4. Fc receptors were blocked with anti-mouse CD16/CD32 antibodies (BD Biosciences, San Jose, Calif.). PE-Ly6G and -CD8, FITC-Ly6C (BD Biosciences), APC-CD11b, Pacific Blue-CD4, and eFlour 450-F4/80 (eBioscience, San Diego, Calif.), and PE/Cy7-CD11c (Biolegend, San Diego, Calif.) anti-mouse mAb, and respective isotype controls were used. Total cells were gated to obtain the percent of myeloid (CD11b⁺), macrophage (CD11b⁺F4/80⁺), DC (CD11b⁺CD11c⁺) and MDSCs (CD11b⁺Ly6G⁺ and CD11b⁺Ly6C⁺) based on specific isotype controls. After gating on CD11b⁺ cells, the proportion of granulocytic MDSCs (Ly6G⁺Ly6C^{low}) and monocytic MDSCs (Ly6C⁺Ly6G⁻) was determined Effector OT-I cells were analyzed using surface staining for PerCP-CD8 (Biolegend), APC-Cy7-Thy 1.1 and PECy7-CD107α (BD Biosciences), in addition to intracellular staining for PE-Granzyme B (eBioscience) and APC-IFN-γ (BD Biosciences) anti-mouse mAb. Cells were stimulated with SIINFEKL (3 μM for 1 h), followed by incubation with Brefeldin A (300 μg/ml for 4 h) for intracellular staining. Cells were fixed and permeabilized using the Cytofix/Cytoperm kit following manufacturer's instructions (eBioscience).

Myeloid cell-mediated immunosuppression was evaluated based on suppression of stimulated T cell proliferation in co-culture experiments using known techniques. Peritoneal myeloid cells and macrophages from tumor-bearing mice were column-purified with anti-CD11b and anti-F4/80 magnetic beads, respectively, using autoMACS according to the manufacturer's protocol (Miltenyi Biotec Inc., Auburn, Calif.). Following column separation, the purity of cell fractions was analyzed microscopically by Diff-Quick-stained cytospins (Fisher Scientific) and by flow cytometry (˜90%). Splenocytes from non-tumor-bearing C57BL/6 female mice were used as naive T cell targets. Following RBC lysis and washing, splenocytes were incubated with 5 μM carboxyfluoresceindiacetate succinimidyl ester (CFSE; Invitrogen, Grand Island, N.Y.) in PBS for 8 min using known methods. Cells (2.5×10⁵ cells/well) were cultured in triplicate in 96-well plates coated with anti-CD3 (10 μg/m1) mAb (BD Biosciences) and B7.1 antigen (0.5 μg/ml) (R&D Systems Inc., Minneapolis, Minn.). Equal numbers of magnetically separated CD11b⁻, CD11b⁺ or F4/80⁺ cells isolated from tumor-bearing mice were added. After 72 hours of co-culture, cells were collected, labeled with anti-CD4 and anti-CD8 mAb, and analyzed by flow cytometry. The proliferation of CFSE-labeled CD4⁺ and CD8⁺ T cells was evaluated by quantification of CFSE dilution. The primary endpoint was the proportion of CFSE-loaded CD4⁺ and CD8⁺ T cells undergoing ≧1 replication.

To evaluate whether peritoneal macrophage-mediated suppression of T cell proliferation was contact-dependent, transwell assays were performed. The myeloid cell functional assay, described above in 96-well plates, was modified with a five-fold increase in the number of cells and plated in 24-well plates using the transwell system (VWR International, Bridgeport, N.J.). PECs collected from non-tumor bearing C57BL/6 female mice were purified with anti-CD11b magnetic beads using autoMACS according to the manufacturer's protocol (Miltenyi Biotec Inc.). Splenocytes from non-tumor bearing mice (2×10⁶ cells/well) were plated in the wells of the 24-well companion plate in contact with stimulus (anti-CD3 and B7.1), while equal numbers of CD11b⁺ PECs were added in the chamber of the transwell cell culture insert (0.4 μm). After 72 hours of co-culture, cells were collected and analyzed by flow cytometry to measure the proliferation of CFSE-labeled CD4⁺ and CD8⁺ T cells based on quantification of CFSE dilution staining as described.

Myeloid cells in ascites of patients with newly diagnosed advanced EOC: Ascites (50 ml) was collected for research at the time of primary surgery in patients with newly diagnosed stage III EOC under an IRB-approved protocol. All subjects signed informed consent prior to surgery. PECs were subjected to RBC lysis with ACK buffer, followed by washing. PECs were either used within 24 h of harvest for flow cytometry and functional studies or frozen in liquid nitrogen in media containing 20% FBS and 5% DMSO. PE-Cy5-CD33, PE-CD14 (Beckman Coulter, Brea, Calif.), BV 412-CD11b (Biolegend), FITC-HLA-DR (BD Biosciences), anti-human mAb and respective isotype controls were used to analyze surface molecule expression and sorting of human PECs. In addition, PE-Cy7-HLA-DR (BD Biosciences), APC-CD14, and FITC-CD15 (Invitrogen) anti-human mAb were used to evaluate the proportion of macrophage and MDSC in PECs from EOC patients.

To evaluate the immunosuppressive properties of peritoneal myeloid cells, PECs were

FACS-sorted to isolate macrophages (CD11b⁺CD33⁺CD14⁺DR⁺) and granulocytic cells (CD11b⁺CD33⁺CD14⁻DR⁻CD15⁺). The purity of the post-sort cell population was confirmed by flow cytometry (>90%). Normal donor allogeneic CD4⁺ and CD8⁺ T cells were used as responders in co-culture experiments using established methods. T cells were purified with anti-CD4 and anti-CD8 magnetic beads using autoMACS according to the manufacturer's protocol (Miltenyi Biotec Inc.), and preserved in liquid nitrogen with 20% FBS and 5% DMSO in complete media. Freshly isolated PECs (macrophages, MDSCs or non-myeloid CD11b⁻CD33⁻ cells) from patients were incubated in triplicate in 96-well round-bottom plates for 4 days with equal numbers (1×10⁵) of normal donor T cells (CD4⁺ and CD8⁺). CD3/CD28 Dynabeads (2 μl) (Invitrogen) were added to each well to activate T cell proliferation. T cell proliferation was measured by [³H]-thymidine (1 μCi per well) incorporation for the final 18 hours of culture. Results are expressed as net counts per minute (cpm) [average cpm from mixed cultures of T cells with PECs in presence of CD3/CD28—(average cpm from parallel cultures without T cells in presence of CD3/CD28 +cpm from T cells cultures only without CD3/CD28)].

Statistical Analysis: Time to euthanasia was plotted using Kaplan-Meier curves and analyzed using the log-rank method. Comparisons between two groups were assessed by the Mann-Whitney test, and the Kruskal-Wallis test was used for multiple group comparisons. Statistical analysis was performed using Graph Pad Prism 6 software.

While the invention has been described through specific embodiments, routine modifications will be apparent to those skilled in the art and such modifications are intended to be within the scope of the present invention.

What is claimed is:

1. A method for enhancing the effect of an anti-cancer agent comprising concurrently or sequentially administering to an individual who is in need of therapy for a tumor i) the anti-cancer agent; and ii) an agent that causes depletion of myeloid cells and/or inhibits recruitment of myeloid cells to the tumor, wherein the effect of the anti-cancer agent on the tumor is greater relative to the effect of the anti-cancer agent in the absence of the agent of ii).

2. The method of claim 1, wherein the myeloid cells are macrophages or granulocytes, or a combination thereof.

3. The method of claim 1, wherein the individual is in need of therapy for an ovarian tumor.

4. The method of claim 1, wherein the agent of ii) is a monoclonal antibody that specifically recognizes the myeloid cells.

5. The method of claim 4, wherein the monoclonal antibody is selected from an anti-CD11b mAb and an anti-CD33 mAb.

6. The method of claim 1, wherein the anti-cancer agent is an anti-tumor vaccine.

7. The method of claim 1, wherein the anti-cancer agent is MIS416.

8. The method of claim 1, wherein the individual is in need of therapy for an ovarian tumor, wherein the anti-cancer agent is MIS416 and the agent of ii) is selected from anti-CD11b mAb and anti-CD33 mAb.

9. A method for determining whether or not an individual is a candidate for a tumor therapy comprising i) an anti-cancer agent and ii) an agent that causes depletion of myeloid cells and/or inhibits recruitment of myeloid cells to the tumor, the method comprising determining whether the individual has a tumor characterized by undesirable myeloid cell proliferation and/or tumor infiltration and/or myeloid cell recruitment to the tumor, and if such determination is made, designating the individual as a candidate for the therapy.

10. The method of claim 9, wherein the individual is determined to have an ovarian tumor.

11. The method of claim 10, further comprising administering to the individual sequentially or concurrently the anti-cancer agent and the agent of ii).

12. The method of claim 11, wherein the anti-cancer agent of ii) is a monoclonal antibody that specifically recognizes the myeloid cells.

13. The method of claim 12, wherein the monoclonal antibody is selected from an anti-CD11b mAb and an anti-CD33 mAb.

14. The method of claim 12, wherein the anti-cancer agent is an anti-tumor vaccine.

15. The method of claim 12, wherein the anti-cancer agent is MIS416.
